SEQUENCE LISTING (12 sequences)

SEQ ID NO: 1 - DNA, 38 bp, artificial sequence
  acceptor and donor regions depicted in Figures 4B and 5B
gaattctaatacgactcactataggaagagatggcgac

SEQ ID NO: 2 - DNA, 33 bp, artificial sequence
  template region depicted in Figures 4B and 5B
gtcgccatctcttcctatagtgagtcgtattag

SEQ ID NO: 3 - DNA, 15 bp, artificial sequence
  primer 1 binding site depicted in Figures 4B and 5B
agctgatcctgatgg

SEQ ID NO: 4 - DNA, 25 bp, artificial sequence
  primer 3 binding site depicted in Figure 6B
gtgccaagcttaccgactcactata

SEQ ID NO: 5 - DNA, 17 bp, artificial sequence
  loop depicted in Figure 6B
ggcagctgatcctgatg

SEQ ID NO: 6 - DNA, 15 bp, artificial sequence
  primer 5 binding site depicted in Figure 6B
actgctgattcgatg

SEQ ID NO: 7 - DNA, 46 bp, artificial sequence
  self-adenylating deoxyribozyme substrate depicted in Figure 6B
ggaagcatagcgacggcgggagaaggggggttggggtctcgagaac

SEQ ID NO: 8 - DNA, 97 bp, artificial sequence
  depicted in Figure 7
gtgccaagcttaccgactcactatagtcgctgagccaagggtcggtttcagaaatcatatcgcttctagcgacggtctaccgttcgcaggctcgcag

SEQ ID NO: 9 - DNA, 19 bp, artificial sequence
  L208 self-ligating deoxyribozyme depicted in Figure 7
atgactgctgattcgatga

SEQ ID NO: 10 - DNA, 26 bp, artificial sequence
  primer 3 used in conjoined polynucleotides in Example 2
gtgccaagcttaccgactcactatag

SEQ ID NO: 11 - DNA, 46 bp, artificial sequence
  primer 4 used in conjoined polynucleotides in Example 2
gttctcgagaccccaaccccccttctcccgccgtcgctatgcttcc

SEQ ID NO: 12 - DNA, 15 bp, artificial sequence
  n = r(U); used in conjoined polynucleotides in Example 2
catcgaatcagcagn

(19) United States
(12) Patent Application Publication
(10) Pub. No.: US 2005/0227331 A1
(43) Pub. Date: Oct. 13, 2005

(54) Conjoined polynucleotide catalysts

(75) Inventor: Ronald R. Breaker, Guilford, CT (US)

Correspondence Address: Arthur G. Schaier;Carmody & Torrance, LLP, 50 Leavenworth Street, P.O. Box 1110, Waterbury, CT, 06721-1110, US

(73) Assignee: Yale University

(21) Appl. No.: 11/054,106

(22) Filed: Feb. 9, 2005

Related U.S. Application Data
(63) Continuation of application No. 10/168,038, filed on Apr. 9, 2002, now abandoned.
(60) Provisional application No. 60/159,808, filed on Oct. 15, 1999.

(51) Int. Cl. C07H 21/02; C07H 21/04; C12P 19/28

(52) U.S. Cl. 435/85; 536/23.1

(57) ABSTRACT

Conjoined polynucleotides are linked RNA and/or DNA that comprise at least tow catalytic domains which function in concert to provide a chemical transformation involving multiple sequential or component reactions. In many embodiments, the domains are fused together, typically by means of conventional 3′→5′ phosphodiester bonds, with or without intervening nucleotides which are not part of the catalytic domains per se, to form conjoined polynucleotides using standard ligation procedures. Conjoined DNA comprising kinase and adenylase domains and conjoined polynucleotides comprising kinase; adenylase, and ligase domains are useful in DNA cloning.

### CROSS-REFERENCES TO RELATED APPLICATIONS

This application claims priority benefit of co-pending U.S. application Ser. No. 60/159,808, filed Oct. 15, 1999, which is incorporated herein in its entirety by reference.

### STATEMENT REGARDING FEDERALLY SPONSORED RESEARCH

The invention was made with partial government support under NIH grant GM57500. The government has certain rights in the invention.

### BACKGROUND OF THE INVENTION

1. Field of the Invention

This invention relates primarily to the development and use of polynucleotide constructs that carry more than one catalytic domain and, by their specific design and functional action in concert, achieve more complicated chemical tasks than do individual ribozymes or deoxyribozymes.

2. Description of Related Art

Enzymes that are made of protein dominate biocatalysis in modern organisms. These enzymes speed the rates of chemical reactions by up to 17 orders of magnitude. Over the last several years, it has become apparent that RNA and DNA also have substantial propensity for molecular recognition and catalysis. The discovery of RNA enzymes or “ribozymes”, made nearly two decades ago, initiated a major reorganization of the widely held doctrine of biocatalysis that had been central to understanding the origin, evolution, and processes of all life. It is now well-accepted that life first began with organisms comprised solely of RNA, that ribozymes functioned as the first enzymes, and that protein and DNA became important only much later as more complex forms of life evolved (Gesteland, R. F., et al., 1999, The RNA World, Cold Spring Harbor Laboratory Press, Cold Spring Harbor, New York; this and subsequent papers and patents cited hereafter are expressly incorporated herein in their entireties by reference). Ribozymes catalyze a variety of chemical reactions including, but not limited to, RNA cleavage, RNA ligation, RNA phosphorylation, RNA adenylation, DNA cleavage, peptide bond formation, formation of other amide bonds, aminoacylation, alkylation, metalation of porphrin rings, transesterification, and isomerization (Breaker, R. R., 1997, Chem. Rev. 97, 371-390).

More recent studies have provided substantial evidence that DNA (functioning as “deoxyribozymes”) also can catalyze reactions of biological significance. Single-stranded DNA is capable of forming intricate tertiary structures that can bind various ligands and promote chemical transformations (Breaker, R. R., 1997, Nature Biotechnol. 15, 427-431). Although catalytic DNAs have not been found in nature, new deoxyribozymes are being isolated from random-sequence pools by using various in vitro selection protocols (Breaker, R. R., 1997, Curr. Opin. Chem. Biol. 1, 26-31; Sen, D. & Geyer, C. R., 1999, Curr. Opin. Chem. Biol. 4, 579-593; Li, Y. & Breaker, R. R., 1999, Curr. Opin. Struct. Biol. 9, 315-323; and Breaker, R. R., 1999, Nature Biotechnol. 17, 422-423). The catalytic repertoire of DNA encompasses some of the same reactions that are catalyzed by ribozymes. These include DNA self-processing reactions and catalysis of various modification reactions on separate nucleic acid substrates. Using in vitro selection from random sequence pools, for example, deoxyribozymes that exhibit oxidative cleavage of DNA (Carmi, N., et al., 1996, Chem. Biol. 3, 1039-1046 and Carmi, N., et al., 1998, Proc. Natl. Acad. Sci. USA. 95, 2233-2237), joining of chemically activated DNAs (Cuenoud, B. & Szostak, J. W., 1995, Nature 375, 611-614), metalation of porphyrins (Li, Y. & Sen, D., 1996, Nat. Struct. Biol. 3, 743-747), DNA phosphorylation (Li, Y. & Breaker, R. R., 1999, Proc. Natl. Acad. Sci. USA. 96, 2746-2751), DNA adenylation (Li, Y., et al., 2000, Biochemistry 39, 3106-3114), and DNA ligation (described below) have been isolated. In addition, cleavage of RNA by divalent metal-dependent, histidine-dependent, and cofactor-independent deoxyribozymes has recently been reported (Li & Breaker, 1999 Curr. Opin. Struct. Biol., cited above). Moreover, the catalytic efficiency exhibited by many deoxyribozymes is comparable to that of ribozymes with identical or related catalytic activities.

Using new methods of rational and combinatorial design, the functional repertoire of RNA and DNA beyond that observed in nature continues to expand (Breaker, Chem. Rev. cited above). Some of the chemical transformations that can be promoted by newly created ribozymes and deoxyribozymes are of significance because they could be applied in various biotechnology or industrial applications. For example, new RNA-cleaving enzymes are of interest due to their potential utility as anti-mRNA and anti-viral agents (Breaker, R. R., 1999, Nat. Biotechnol. 17, 422-423 and Christofferson, R. E. & Marr, J. J., 1995, J. Med. Chem. 38, 2023-2037). In addition, RNA and DNA “aptamers” that have specific ligand-binding functions that are being developed as diagnostic and therapeutic agents (Gold, L., et al., 1995, Annu. Rev. Biochem. 64, 763-797 and Osborne, S. E. & Ellington, A. D., 1997, Chem. Rev. 97, 349-370).

Except for a recent report of the in vitro evolution of a bifunctional ribozyme which recognizes an activated glutaminyl ester and aminoacylates a target tRNA (Lee, N., et al., 2000, Nature Struct. Bio., 7, 28-33), previous reports on the catalytic activities of ribozymes and -deoxyribozymes have employed them exclusively in isolation without combining the catalytic features of more than one enzyme to enhance their overall chemical sophistication. For example, self-ligating ribozymes have been generated (Bartel, D. P. & Szostak, J. W., 1993, Science 261, 1411-1418 and Robertson, M. P. & Ellington, A. D., 1999, Nat. Biotechnol. 17, 62-66) as have self-ligating deoxyribozymes (described below). However, each of these enzymes requires a chemically activated substrate that is supplied to the reaction mixture by the experimenter. No one has discussed or demonstrated the concept of combining catalytic domains such that a single or several polynucleotide chains carry out a set of sequential reactions in a serial or parallel fashion. This arrangement, wherein conjoined RNA or DNA catalyze a set of chemical reactions, makes possible the ability to achieve far more complicated tasks.

### BRIEF SUMMARY OF THE INVENTION

It is an objective of the invention to judiciously combine RNA or DNA enzymes (either natural or engineered) to create conjoined polynucleotides that have higher-ordered functions.

It is a more specific objective of the invention to provide polynucleotide constructs and complexes that are conjoined RNA and/or DNA catalysts which have multiple catalytic domains that accelerate more than one chemical reaction.

These and other objectives are accomplished by the present invention, which provides polynucleotide RNA, DNA, or mixed RNA and DNA combinations, that comprise at least two catalytic domains which function in concert to provide a chemical transformation involving multiple sequential or component reactions. In many embodiments, the domains are fused together, typically by means of conventional 3′→5′ phosphodiester bonds, with or without intervening nucleotides which are not part of the catalytic domains per se, to form constructs using standard ligation procedures.

Conjoined polynucleotide of the invention are particularly useful for carrying out sequential polynucleotide manipulations in a serial fashion. For example, an embodiment illustrated hereafter describes the preparation of polynucleotide constructs of the invention that comprise a kinase catalytic domain and an adenylase catalytic domain that respectively phosphorylate and adenylate reactions of a polynucleotide substrate in the presence of ATP to form a phosphorylated and adenylated product. Primarily using ribozyme and deoxyribozyme in vitro selection techniques previously described, a variety of catalytic RNAs and DNAs have been produced which phosphorylate themselves or target polynucleotides, forming a terminal phosphate, and others which catalyze the transfer of an AMP moiety of ATP to the terminal phosphate group, forming a cap. These include classes of DNAs that “self-phosphorylate” (Li & Breaker, 1999 P.N.A.S., cited above) and others that “self-adenylate” (Li, et al., 2000 Biochemistry, cited above). In the practice of this embodiment of the invention, these domains are conjoined to form a construct that has both catalytic domains such that both chemical reactions are carried out in a serial fashion when ATP is present. (A DNA construct is illustrated in FIG. 2). The polynucleotide constructs thus comprise a kinase domain that catalyzes the phosphorylation of a 5′-hydroxyl group on a ribose or 2′-deoxyribose moiety of a polynucleotide substrate in the presence of ATP to form a 5′-terminal phosphate group on the substrate, and an adenylase domain that catalytically transfers, in the presence of ATP, the AMP moiety of an ATP to the 5′-terminal phosphate group on the substrate (FIG. 1A), to form a phosphorylated and adenylated, or “capped”, product (FIG. 1B). The polynucleotide substrate so phosphorylated and adenylated can be the polynucleotide construct itself, a polynucleotide covalently linked to the construct, or another polynucleotide.

Since the overall process transfers chemical energy stored in the phosphoanhydride bonds of ATP by formation of the 5′ cap, the phosphorylated and adenylated chemically activated product can serve as a “power generator” for subsequent reactions. In some preferred embodiments, polynucleotide constructs of the invention that have both kinase and adenylase domains further comprise a polynucleotide ligase domain that catalyzes, in the presence of ATP, the ligation of the phosphorylated, adenylated product to target oligonucleotides, forming a new 3′,5′-phosphodiester bond. (The reaction is illustrated in FIG. 1C and a construct, in FIG. 3.) In alternate embodiments, the ligase domain physically conjoins with the kinase/adenylase construct to achieve the same reaction sequence, and in some embodiments all three domains are physically rather than chemically conjoined. The oligonucleotide target for the ligase reaction can be the construct itself, the construct covalently attached to another polynucleotide, or another polynucleotide.

Conjoined DNA of the invention described more fully below comprise domains that catalyze the three deoxyribozyme activities illustrated in FIG. 1, which are chemical steps used most cloning strategies. Using conventional methodology that employ protein enzymes, DNA phosphorylation is achieved using the enzyme T4 polynucleotide kinase. This DNA phosphorylation activity is requisite, but not sufficient, for enzymatic ligation of unphosphorylated DNA oligonucleotides that is used in most DNA cloning processes. The next preparative step for conventional DNA cloning is ligation of the DNA of interest into a vector. A separate protein enzyme termed T4 DNA ligase carries out this reaction. This enzyme catalyzes a two-step reaction wherein the DNA is first adenylated or “capped” by AMP (with the use of ATP), then subsequently ligated to the appropriate DNA acceptor. This invention provides all three catalytic functions typically carried out by polypeptides in a single DNA molecule or a single conjoint DNA complex. And, using routine engineering techniques, deoxyribozymes useful in cloning can be programmed to self-circularize or join a separate DNA of a specific sequence for rolling circle or conventional amplification of genes-of-interest. Similarly, RNA constructs and complexes can be generated by combining RNA kinase, RNA adenylase, and RNA ligase catalytic domains.

In alternative embodiments, instead of a ligase domain, other catalytic domains conjoined to the constructs or complexes make use of the energy stored in the cap to, for example, covalently attach to specific targets, such as proteins, oligosaccarides, lipids, synthetic polymers, and the like—all in a process that is similar to DNA cloning described above.

### DESCRIPTION OF THE FIGURES

FIG. 1A illustrates the reaction catalyzed by a DNA kinase domain, FIG. 1B, a DNA adenylase domain, and FIG. 1C, a DNA ligase domain of a deoxyribozyme or a DNA construct comprising multiple domains. Together, the three deoxyribozyme activities are important reactions for DNA cloning in some embodiments of the invention illustrated in Examples 1 and 2.

FIG. 2 is a schematic drawing of a DNA construct of the invention comprising kinase and adenylase domains illustrated in Example 1.

FIG. 3 is a schematic drawing of a DNA construct of the invention comprising kinase, adenylase, and ligase domains useful in DNA cloning illustrated in Example 2.

FIG. 4 schematically illustrates the selection protocol for self-phosphorylating deoxyribozymes described in Example 1. (A) Selection is initiated by incubating a population of synthetic 100-nucleotide DNAs that carry a random-sequence domain of 70 nucleotides. DNAs are (I) incubated with a mixture of either the four standard NTPs or the four standard dNTPs. After incubation, the DNA pool is (II) combined with excess acceptor and template oligonucleotides, and treated with T4 DNA ligase in the presence of ATP. The ligated DNAs (˜123 nucleotides) are (III) isolated by PAGE and the recovered DNAs are selectively amplified by PCR using a primer denoted 1 and a ribo-terminated primer denoted 2. The resulting double-stranded PCR products are (IV) treated with NaOH to cleave the isolated RNA linkage, and the subsequent single-stranded DNAs are purified by PAGE and used (V) to initiate the next round. DNA populations from the final rounds of selection were (VI) PCR-amplified with primer 1 and the “all-DNA” version of primer 2 to facilitate cloning. N70 represents the random-sequence domain. (B) Sequences of the DNA population complexed with the acceptor and template strands (SEQ ID NOs 1, 2, and 3).

FIG. 5 schematically illustrates the selection protocol for self-capping deoxyribozymes described in Example 1. (A) A pool of 10¹⁴ synthetic 100mer DNAs, each carrying a 70-nt random-sequence domain and a 5 phosphate is (I) incubated in selection buffer containing ATP. After incubation, the DNAs are separated from ATP by PAGE and the DNA recovered from the gel is (II) combined with excess acceptor and template oligonucleotides and treated with T4 DNA ligase in the absence of ATP. The ligated DNA (≈123 nt) is (III) isolated by PAGE, and the recovered DNAs are selectively amplified by PCR using a primer denoted 1 and a ribo-terminated primer denoted 2. The resulting double-stranded PCR products are (IV) treated with NaOH to cleave the single RNA linkage, and the corresponding single stranded DNAs (≈100 nt) are separated by PAGE. The recovered DNA is (V) phosphorylated with T4 PNK in the presence of ATP, purified by PAGE, and the resulting DNA population is (VI) used to initiate the next round of selection. DNAs from the final round of selection are (VII) PCR-amplified with primer 1 and an “all-DNA” version of primer 2 to facilitate cloning. N₇₀ represents the random-sequence domain. (B) Sequences of the DNA population complexed with acceptor and template oligonucleotides (SEQ ID NOs 1, 2, and 3).

FIG. 6 schematically illustrates the selection protocol for the isolation of self-phosphorylating DNAs described in Example 2. (A) A population of (+) strand random-sequence DNAs is (I) incubated with a chosen source of phosphate (e.g. ATP) under permissive reaction conditions. The resulting DNA products are (II) combined with an appropriate acceptor-template combination that hybridizes to the donor domain of each DNA in the population such that the 5′ end of the donor is adjacent to the 3′-hydroxyl group of the acceptor oligomer within the templated complex. Only those DNAs that have acquired a 5′-phosphate group serve as substrates for T4 DNA ligase, which joins each phosphorylated DNAs to an acceptor oligonucleotide. Ligated DNAs are (III) isolated by PAGE, recovered from the gel by crush/soak isolation, and amplified by PCR. One of the two PCR primers is terminated with a ribonucleotide, which yields double-stranded DNAs wherein the (+) strand of each carries a single embedded RNA linkage (X). Single-stranded (+) DNAs are (IV) generated by incubation of the PCR product with NaOH and the full-length deoxyribozymes are purified by PAGE. The resulting (+) strand DNAs are (V) incubated as described in step I to initiate the subsequent round of selection. Steps II through V are repeated until the DNA population exhibits the desired level of catalytic activity. To examine individual kinase deoxyribozymes, the final population is (VI) amplified with ‘all-DNA’ PCR primers and the resulting double-stranded DNAs are subjected to cloning and sequencing. (B) Representative selection construct described in Example 1 to isolate NTP-dependent self-phosphorylating deoxyribozymes. N₇₀ represents a domain of 70 random-sequence nucleotides (SEQ ID NOs 4, 5, 6, and 7).

FIG. 7 illustrates the sequence and secondary structure of a self-ligating DNA that requires the action of a self-phosphorylating DNA for its function (more fully described in Example 2; SEQ ID NOs 8 and 9). The L208 deoxyribozyme catalyzes the formation of a 3′,5′-phosphodiester linkage between the 3′-terminus of the ligase deoxyribozyme and the 5′-phosphodiester linkage of a 46-nucleotide self-adenylateing deoxyribozyme (AppDNA). Roman numerals identify structural elements within the 208mer ligase deoxyribozyme. The inset provides gel patterns showing self-ligation activity of the L208 deoxyribozyme. 5′-³²P-labelled L208 incubated with saturating amounts of AppDNA that had been pre-reacted with ATP. “Pre” identifies the precursor L208 deoxyribozyme and “Lig” identifies the conjoined deoxyribozymes that result upon the synergistic function of the separate deoxyribozyme domains.

### DETAILED DESCRIPTION OF THE INVENTION

This invention is based upon the concept of judiciously combining natural or engineered RNA or DNA enzymes to create new constructs or complex-es that have higher-ordered functions. Conjoined polynucleotide catalysts of the invention can be defined one or more polynucleotides that, when chemically and/or physically and/or spatially linked together, carry more than one catalytc domain and, by their specific design, achieve more complicated chemical tasks than typically do individual ribozymes and deoxyribozymes. In many respects, conjoined polynucleotides can be thought of as “molecular machines” whose catalytic domains serve as molecular components or modules (much like the parts of a mechanical device) that each serve a specified function. In certain embodiments, conjoined polynucleotides of the invention are structurally and functionally similar to viruses. However, constructs and complexes of the invention can be assembled from component polynucleotide catalytic domains, whereas viruses are usually reverse engineered to provide useful tools for therapeutics and biotechnology. In contrast to the use of natural viruses, conjoined polynucleotides can be engineered to catalyze reactions or bring about some other desired effect that are entirely defined by the user.

As summarized above, this invention broadly encompasses polynucleotide constructs and complexes which have at least two catalytic domains which function in concert to provide a chemical transformation involving multiple sequential or component reactions. Catalytic domains in the constructs and complexes are polynucleotide sequences that increase the rate or efficiency of a chemical reaction, including sequences that enhance or stimulate the efficiency of other catalytic sequences (sometimes referred to as “catalyst promoters”), which are comprised of any natural, recombinant, or synthetic RNA, DNA and mixtures of RNA and DNA, including sequences that have RNA and/or DNA analogues. Analogues include chemically modified bases and unusual natural bases such as, but not limited to, 4-acetylcytidine, 5-(carboxyhydroxymethyl)uridine, 2′-O-methylcytidine, 5′-carboxymethylaminomethyl-2-thioridine, 5-carboxymethyl-aminomethyluridine, dihydrouridine, 2′-O-methylpseudouridine, β-D-galactosyl-queosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methylguanosine, 2-methyladenosine, 3-methylcytidine, 5-methylcytidine, N6-methyladenosine, 7-methylguanosine, 5-methylaminomethyluridine, 5-methoxyaminomethyl-2-thiouridine, β-D-mannosylqueosine, 5-methoxycarbonylmethyluridine, 5-methyloxyuridin, 2-methylthio-N6-isopentenyladenosine, N((9-β-D-ribofuranosyl-2-methylthiopurine-6-yl)carbamoyl)threonine, N-((9-β-D-ribofuranosylpurine-6-yl)N-methyl-carbamoyl)threonine, uridine-5-oxyacetic acid methyl ester, uridine-5-oxyacetic acid, wybutoxosine, pseudouridine, queosine, 2-thiocytidine, 5-methyl-2-thiouridine, 2-thiouridine, 4-thiouridine, 5-methyluridine, N-((9-β-D-ribofuranosylpurine-6-yl)carbamoyl)threonine, 2′-O-methyl-5-methyluridine, 2′-O-methyluridine, wybutosine, and 3-(3-amino-3-carboxypropyl uridine. (See also Eaton, B. E., & Piekin, W. A., 1995, Annu. Rev. Biochem. 64, 837-863.) Further encompassed by the invention are polynucleotides modified during or after preparation of the domains and constructs using standard means. DNA and/or RNA starting materials for the domains, and constructs and complexes containing them, may be isolated from whole organisms, tissues or tissue cultures; constructed from nucleotides and oligonucleotides using standard means; obtained commercially; selected from random and enriched in vitro or in vivo sequence pools; and combinations thereof.

Constructs and complexes of the invention are prepared by chemically and/or physically and/or spatially linking together catalytic domains which function in concert to provide a chemical transformation involving multiple sequential or component reactions. Constructs may be linear or branched, linked together such that the catalytic domains are positioned in proximity and under conditions sufficient for sequential reaction to occur, i.e., so that reaction substrates and intermediate products are suitably exposed to the polynucleotide active sites to provide a setting for the dynamic exchange of structures that occurs during the transformation. Complexes are similarly grouped to bring the polynucleotide catalytic domains into contact or near contact for sequential reactions to occur. Some chemical transformations are catalyzed by both constructs and complexes; others, including one illustrated in Example 2, involve a construct and one or more ribozymes and/or deoxyribozymes. The maximum number of domains depends upon the number of steps necessary to achieve the desired transformation, the size of the catalytic domains, and their arrangement and efficiency. The examples that follow illustrate conjoined polynucleotides comprising two or three domains, but species containing about 4 to seven domains are necessary for some transformations, and others containing many more domains can be designed for sequential reactions because of the flexibility and size of typical polynucleotides. Moreover, as will be discussed more fully below, the selection method for generating useful domains can generate polynucleotides of varying size and complexity. Thus, the overall structure of conjoined polynucleotides of the invention is determined by their function, which will allow the component or sequential reactions to proceed for a time and under conditions sufficient to generate the intermediate and final products required for the overall transformation.

Most construct embodiments of the invention employ conventional 3′→5′ phosphodiester bonds, with or without intervening nucleotides which are not part of the catalytic domains per se, to link the domains using standard biochemical ligation procedures or chemical bonding, but the invention also encompasses constructs that link the catalytic domains with non-nucleotide moieties or bridges. Constructs are prepared either by fusing pre-existing ribozyme or deoxyribozyme molecules or by fusing random-sequence domains to a pre-existing polynucleotide enzymes. In either case, the domains can be coupled, for example, with DNA ligase in combination with a template oligonucleotide. Alternatively, DNA polymerase or reverse transcriptase can fuse the two hybridized domains using a 3′ extension reaction. In some circumstances, functional conjoined polynucleotides can be prepared by fusing a pre-existing functional domain to a random-sequence domain. In vitro selection then provides a conjoined complex that exhibits the desired multifunctional activity.

Conjoined polynucleotide complexes are prepared by mixing multiple polynucleotide catalysts, by physically attaching polynucleotide catalysts to each other, e.g., using Watson-Crick base pairing or other means, by dispersing catalytic polynucleotides in an ordered matrix, and/or by containing them together in liposomes or other delivery vehicle. In many embodiments, individual deoxyribozymes and/or and/or constructs are physically or chemically immobilized on a support such as, but not limited to, beads, slides, and columns, using standard means. Alternatively and preferably, DNAs or RNAs that catalyze the reactions necessary to couple to specific compounds, e.g., by use of the conjoined kinase/adenylase complex or construct with a nucleophile, are fused to the catalytic domains or constructs, so that the deoxyribozymes or constructs immobilize themselves. The domains may be linked to other nucleotide moieties or other moieties, and mixtures of domains and/or constructs may contain other chemicals including other polynucleotides.

The catalytic domains are selected to provide rate enhancement of both serial and parallel functions in chemical transformations, and reaction sequences that comprise reaction steps that are performed partially serially and partially in parallel. In many preferred embodiments, conjoined polynucleotides exhibit synergism in their catalysis of the overall chemical transformation, so that the rate and/or efficiency of the individual reactions catalyzed by the domains is improved over what is obtained using the catalysts separately in a step-wise fashion. The constructs and/or complexes can be engineered so that the distances between active sites are such that, when one site denoted, for example, as site “A,” is actively forming an intermediate product, the next site is positioned so that the product formed in the “A” site is quickly acted upon, thereby enhancing the efficiency of the overall transformation. Encompassed by the invention also are constructs which include domains or modules that act by closing the distance between two catalytic sites when an earlier site in the catalytic sequence is acted upon.

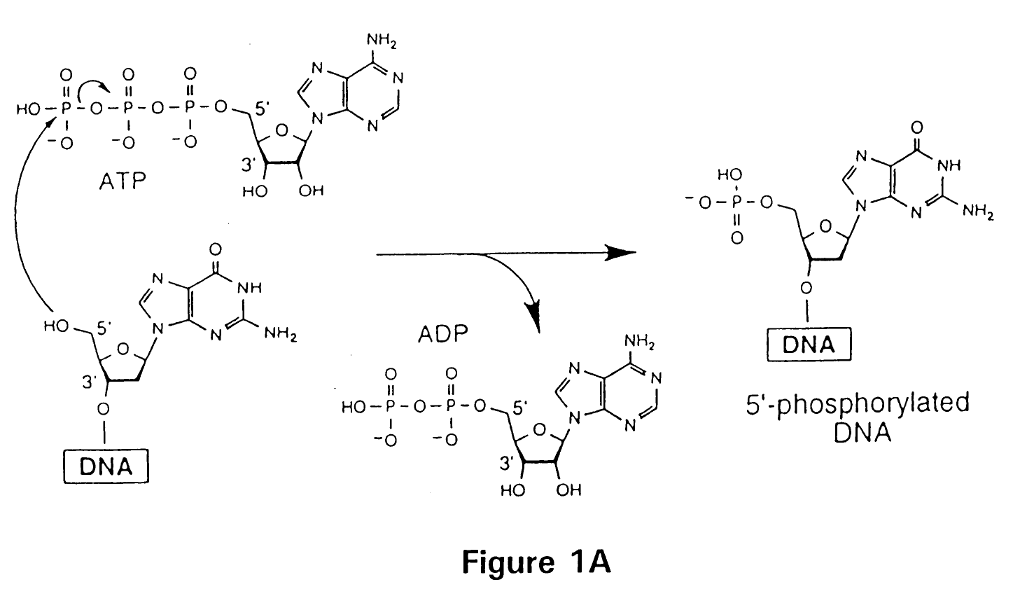

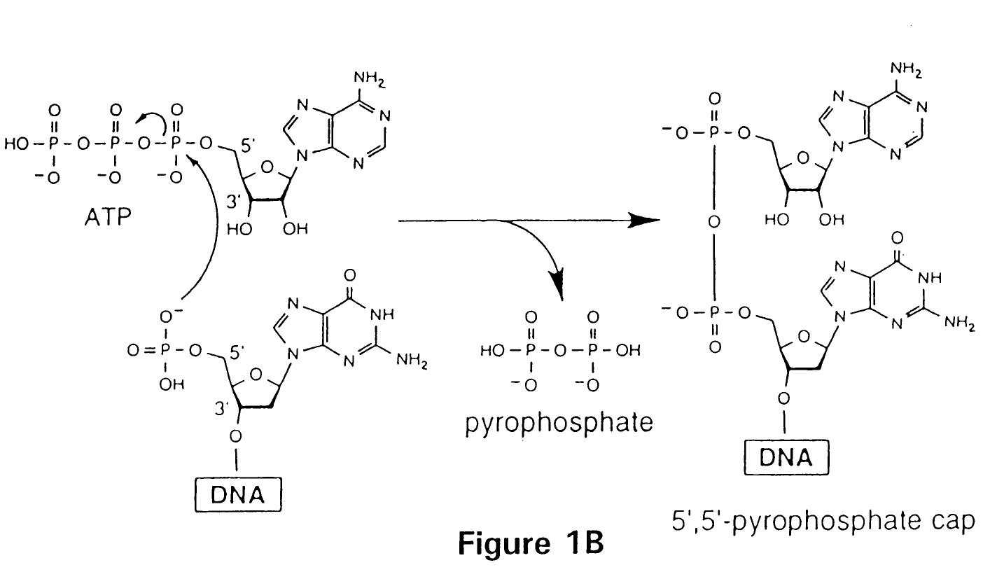

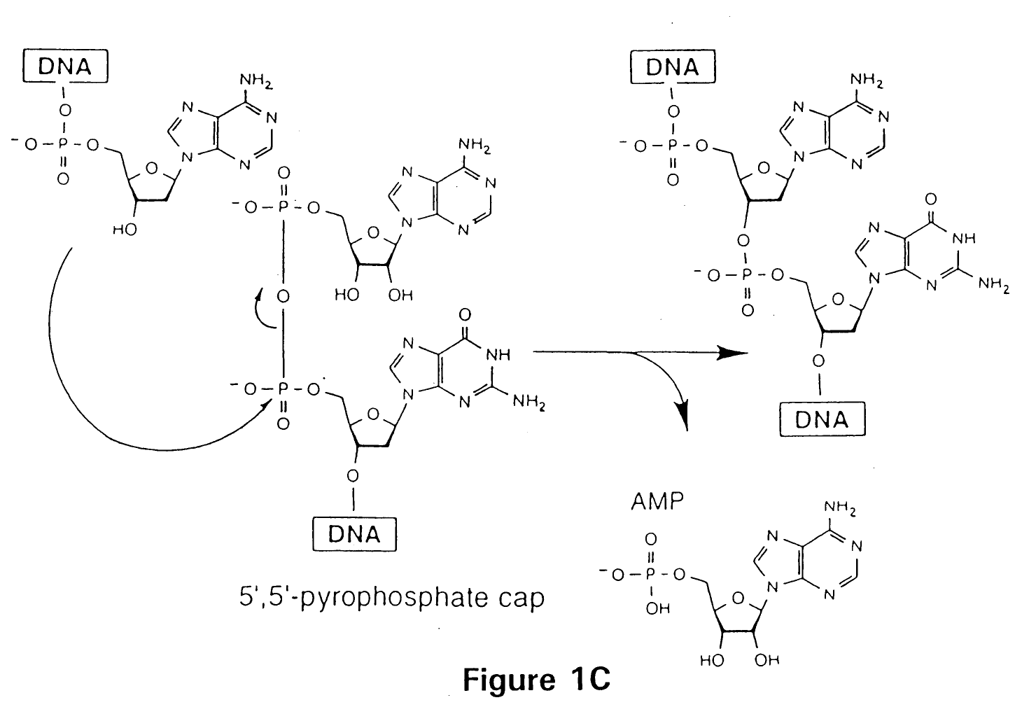

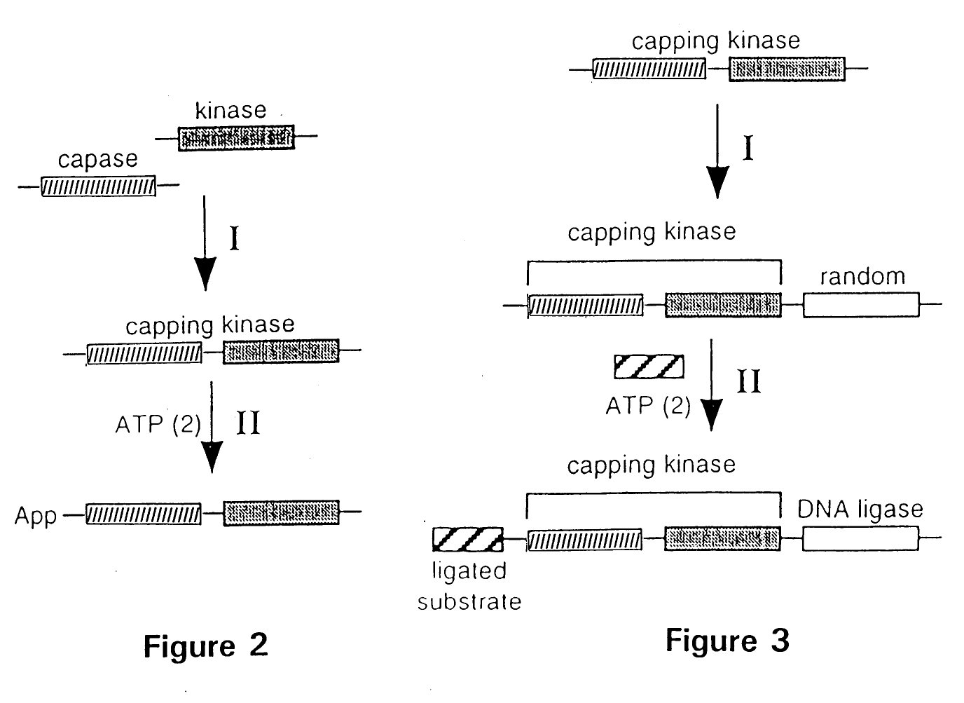

Conjoined polynucleotides of the invention are particularly efficacious for polynucleotide manipulations. Some embodiments illustrated below harness the chemical energy of ATP to power the function of RNA and DNA constructs. For example, a variety of self-phosphorylating DNAs that transfer the γ-phosphate of ATP (or any of the natural (d)NTPs) to their 5′ terminus were prepared (Li & Breaker, 1999 P.N.A.S., cited above). This “DNA kinase” activity is a fundamental reaction for the manipulation of DNA (FIG. 1A). In addition, a series of adenylase deoxyribozymes that efficiently charge themselves at the expense of ATP were prepared (Li, et al., 2000 Biochemistry cited above; see FIG. 1B). These two pre-existing domains are fused into a single contiguous DNA sequence that sequentially phosphorylates and adenylates its 5′ terminus, thereby chemically activating itself for subsequent reactions (FIG. 2).

The self-adenylating deoxyribozyme activates in the presence of ATP and can subsequently serve as substrate of the DNA ligase deoxyribozyme. Therefore, the kinase-adenylase construct can be fused to, or associated with, a ligase that accepts a self-adenylating deoxyribozyme as a substrate for the formation of a new 3′,5′-phosphodiester bond. This is accomplished using standard modular rational design and in vitro selection strategies to isolate ligase domains described below, and then engineering a tripartite construct by fusing the three distinct catalytic domains to produce a single construct (FIG. 3), or associating the kinase-adenylase construct with a ligase. Integration of all three domains (kinase, adenylase and ligase) would yield an ATP-dependent DNA construct useful in DNA cloning protocols. Using routine engineering techniques and in vitro selection of ligated products (e.g., separations performed by polyacrylamide gel electrophoresis), linear DNA products can be isolated. Alternatively, the tripartite construct can be programmed to self-circularize using catalytic components that, upon activation by ATP, serve as a template for rolling circle replication, or it can be programmed to join to a separate DNA of a specific sequence. Since individual ribozymes are also known to catalyze related reactions, this same strategy can be applied to create a simple RNA construct with RNA ligase activity.

The invention thus provides polynucleotide constructs comprising a kinase domain that catalyzes the phosphorylation of a 5′-hydroxyl group on a ribose or 2′-deoxyribose moiety of a polynucleotide substrate in the presence of ATP to form a 5′-terminal phosphate group on the substrate, and an adenylase domain that catalytically transfers, in the presence of ATP, the AMP moiety of an ATP to the 5′-terminal phosphate group on the substrate, to form a phosphorylated and adenylated product. It also provides conjoined polynucleotides further comprising a ligase domain that carries out a ligase reaction that ligates, in the presence of ATP, the phosphorylated, adenylated product to the construct, the construct covalently attached to another polynucleotide, or another polynucleotide molecule. These can be RNA and/or DNA.

In alternate embodiments, the combined “phosphorylating/adenylating” construct can serve as a generalized component of various other DNA constructs or complexes linked to other domains. These exhibit other catalytic activities, such as chemical coupling to specific chemical targets, e.g., proteins, oligosaccarides, lipids, synthetic polymers, and the like, and other biological and non-biological agents, particularly those that carry an appropriate nucleophile. One particularly efficacious embodiment employs conjoined polynucleotides in self-labeling reactions. In other examples, classes of conjoined RNA and DNA encompassed by the invention include those engineered by replacing the ligase portion described above with a random-sequence domain or a domain that binds particular protein targets. The construct can then be subjected to in vitro selection to isolate RNA or DNA molecules that utilize ATP (or other chemical agents, e.g., any activated phosphate or substrate the evolved RNA or DNA can accept) to become activated and that subsequently couple to the peptide or protein target. Numerous applications for the resulting polynucleotide-polypeptide fusions are possible, ranging from protein inactivation to protein immobilization and novel protein-RNA or protein-DNA constructions.

This same self-phosphorylating/self-adenylating construct can be linked to a random-sequence domain or to other binding domains for the engineering of constructs that covalently join to any compound that carries an appropriate nucleophile. Suitable nucleophiles are those that can attack a phosphorus center of the activated phosphoanhydride linkage, such as oxygen, nitrogen, sulfur, or other chemical group that can attack the phosphorus center of the adenyl linkage. Alternatively, activation of the RNA or DNA sequence by other chemical agents would further broaden the ability of constructs to carry out chemical coupling reactions. This capability will allow RNA and DNA constructs to covalently couple to a variety of chemical agents so that additional functions can be carried out with the conjoined agent.

As mentioned above, RNAs and DNAs that catalyze the reactions necessary to couple to specific compounds that are immobilized on surfaces will themselves become immobilized. Therefore, conjoined polynucleotides can be engineered to selectively attach to the surfaces of solid supports such as chromatographic matrices, biochips, and molecular assemblies. New RNA and DNA constructs can be created that perform tasks that involve reactions other that chemical coupling. For example, the class II DNA-cleaving DNA can be used as a component of the self-ligating construct described above such that the first three catalytic domains (kinase, adenylase, ligase) work to join two DNAs together, whereupon the fourth domain (class II DNA-cleaving deoxyribozyme) becomes activated and effects a site-specific cleavage of the ligated DNA target. Additional types of constructs could be made using existing deoxyribozymes (such as those discussed in Li & Breaker, 1999 Curr. Opin Struct. Biol., cited above), and other deoxyribozymes that cleave RNA, metalate porphyrin rings, depurinate DNA, that exhibit allostery (such as those described in WO 98/27104 by Breaker, 1998, and WO 00/26226 by Breaker and Soukup, 2000), etc., or that use entirely new catalytic functions that are generated by in vitro selection. An advantage of the invention is that the catalytic domains are modular units that can be assembled in a variety of ways with links that are physical, chemical, and/or spatial to each other or to a support, which can be changed to accomplish different chemical transformations and modified to contain modules or domains that speed the processing of reactive intermediates. The structure of RNA and DNA lends itself to the rational design of many conjoined polynucleotides assembled to efficiently act in concert.

Modular rational design and in vitro selection can be employed using protocols similar to those described previously (Breaker, 1997 Chem. Rev., cited above), as well as those illustrated in Examples 1 and 2. In a typical in vitro selection strategy for deoxyribozymes, a single-stranded DNA pool containing up to 10¹⁶ sequence variants is prepared by chemical or enzymatic means and subjected to repeated rounds of selective amplification. The most commonly used approach to in vitro selection involves self-modification, wherein the substrate (or one of the substrates) of a given chemical reaction becomes covalently linked to the deoxyribozyme. DNAs are incubated under “permissive reaction conditions” to allow those DNAs that exhibit the desired catalytic activity to self-process. The chemically altered DNAs are then separated from unmodified DNAs using an appropriate physical partitioning method such as gel electrophoresis or affinity chromatography. For different selections, the separation method employed must be chosen on the basis of the unique physical and/or chemical properties of the altered DNAs. In some cases, the modified DNAs can be selectively amplified in the presence of unreacted DNAs by employing a molecular recognition event (e.g. Watson/Crick base pairing) to identify DNAs that have undergone self-modification.

After the selection step, the isolated DNA molecules typically are amplified using the polymerase chain reaction (PCR). This process of selection and amplification constitutes one round of in vitro selection. In most cases, however, the functional DNAs sought by the experimenter are exceedingly rare compared to the number of inactive molecules. In addition, the selection step is usually somewhat permissive in that non-functional molecules are recovered as “background” along with the few active DNAs. Therefore, repetitive rounds of selection and amplification are usually necessary to recover the DNA molecules of interest.

To proceed to the second round of in vitro selection, double-stranded DNA produced by PCR must be converted into single-stranded DNA in order to preclude antisense inhibition of each deoxyribozyme by its complementary strand. Several methods such as asymmetric PCR or alkaline-mediated cleavage of DNA-RNA chimeras can yield single-stranded DNAs in significant quantities. Successive rounds of in vitro selection might employ progressively more stringent reaction conditions, or even the introduction of mutations, in order to isolate more efficient deoxyribozymes. When the population exhibits a satisfactory level of catalytic activity, individual deoxyribozymes are isolated and examined using standard DNA cloning and sequencing techniques.

Most critical to the success of any in vitro selection effort is the proper choice and careful execution of the various methodologies necessary to isolate and amplify exceedingly small numbers of deoxyribozymes among a background of functionally inert DNAs. For example, in vitro selection frequently yields molecules that have no connection with the desired catalytic activity, but are manifest in the selection due to some unexpected properties. These can be avoided by employing a stringent selection strategy that precludes the amplification of molecules that fail to catalyze the desired chemical reaction.

Protocols generally involve synthesis of oligonucleotides to provide the random-sequence DNAs, DNA selection using a screen based upon the type of catalyst desired, ligation and purification to concentrate selected DNA populations exhibiting catalytic activity, PCR amplication of the selected DNA population, regeneration and selective amplification of select DNAs, removal of inactive DNAs inadequately removed during purification and inadvertenly amplified with select DNAs (if necessary), and iteration of the selective amplification process to achieve a DNA population exhibiting satisfactory catalytic activity. Examples are given hereafter. The in vitro selection protocols described herein can be altered to accommodate different research goals such as the isolation of deoxyribozymes with, for example, improved catalytic rates, higher substrate affinities, or different substrate specificities. An advantage of the selection protocol is that it can be adapted to provide screening methods for isolating numerous deoxyribozymes exhibiting different catalytic activities that are useful when conjoined with one or more others in the practice of the invention. Another advantage is that the screening and amplification steps all employ standard methodology.

Simple designs for RNA and DNA constructs of the invention can readily be applied to various transformations in biotechnology and industry. Conjoined polynucleotides can be engineered to self-assemble molecular components for various chemical, biochemical, electronic, or industrial applications using the few catalytic RNA and domains that are already available. More advanced designs can be applied for therapeutic purposes. Naturally occurring retroviruses have been programmed by evolution to enter host cells, insert its genetic information into the chromosomes of its target, and commandeer the cellular machinery to produce more virus particles. Similarly, RNA and DNA constructs can be engineered to carry out such tasks as tissue-specific cell invasion and subsequent biochemical modification to bring about a desired therapeutic effect. The number of applications would be limited only by the functional capabilities of nucleic acids and their ability to perform in concert in the context of individual ribozymes and deoxyribozymes. The use of modified nucleic acids, proteins, or other chemical polymers with significant catalytic potential could be used to augment the functional capabilities beyond that of RNA and DNA constructs.

It is an advantage of the invention that use of polynucleotides as catalysts offer advantages over protein-based enzymes in cloning and in a number of commercial and industrial processes. Problems such as protein stability, supply, substrate specificity and inflexible reaction conditions all limit the practical implementation of natural biocatalysts. DNA can be engineered to operate as a catalyst under defined reactions conditions. Moreover, conjoined polynucleotides made from DNA are expected to be much more stable and can be easily made by automated oligonucleotide synthesis, and DNA is significantly more resiostant to hydrolytic degradation compared to RNA. In addition, conjoined DNA and RNA may be selected for their ability to function on a solid support and are expected to retain their activity when immobilized.

### EXAMPLES

The following examples are presented to further illustrate and explain the present invention and should not be taken as limiting in any regard.

### Example 1

This example describes the isolation of classes of DNAs with polynucleotide ATP-dependent kinase-like activity (which catalyze the reaction illustrated in FIG. 1A) and classes of DNAs that promote ATP-dependent adenylase activity (illustrated in FIG. 1B) that are useful as conjoined polynucleotides of the invention. Further illustrated is the preparation of a construct illustrated in FIG. 2 by linking the catalytic domains together.

### Preparation of DNA Phosphorylases

Nearly 50 distinct sequence classes of self-phosphorylating deoxyribozymes were isolated from a random-sequence pool containing ≈10¹⁵ single-stranded DNA molecules using in vitro selection summarized above and described in detail by Li & Breaker, 1999 P.N.A.S., cited above. Each self-phosphorylating deoxyribozyme makes use of one or more of the eight standard ribo- or deoxyribonucleoside 5′-triphosphates (NTPs or dNTPs) as a source of activated phosphate. Although most prototypic deoxyribozymes poorly differentiate between the ribose and deoxyribose moieties, further optimization by in vitro selection produced variants that display up to 100-fold discrimination between related NTP and dNTP substrates. An optimized ATP-dependent deoxyribozyme uses ATP >40,000-fold more efficiently than CTP, GTP or UTP. This enzyme operates with a rate enhancement of nearly 1 billion-fold over the uncatalyzed rate of ATP hydrolysis. A bimolecular version of the ATP-dependent deoxyribozyme was further engineered to phosphorylate specific target DNAs with multiple turnover. The substrate recognition patterns and rate enhancements intrinsic to these DNAs are characteristic of naturally occurring RNA and protein enzymes.

In Vitro Selection of Self-Phosphorylating Deoxyribozymes.

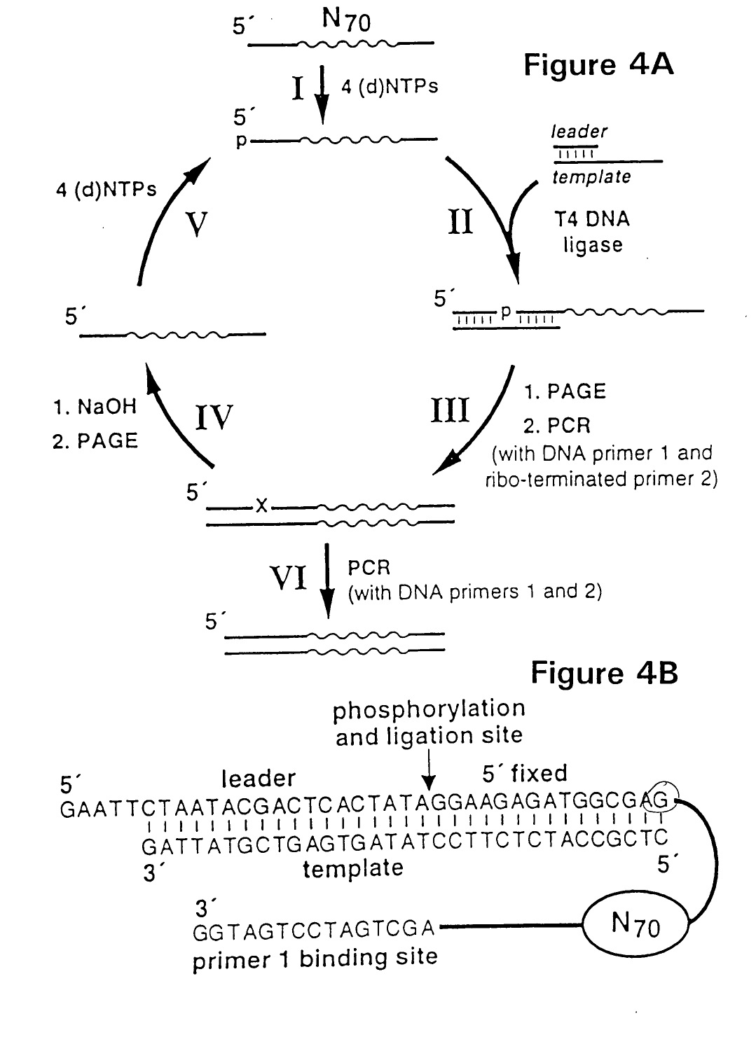

The in vitro selection strategy employed is depicted in FIG. 4. This approach relies on the ability of T4 DNA ligase to join an “acceptor” DNA to a 5′-phosphorylated “donor” DNA when the two are properly juxtaposed within a duplex. Therefore, only those DNAs that acquire a phosphate at any step preceding the ligation reaction will be joined to a DNA acceptor molecule (FIG. 4B). This coupling event distinguishes the reacted DNAs both during PAGE and during selective PCR amplification.

Two parallel selection efforts were undertaken, each with a DNA population comprised of ˜6×10¹⁴ different molecules. The populations were supplied with the four standard ribonucleoside 5′-triphosphates (NTP selection) or the four standard deoxyribonucleoside 5′-triphosphates (dNTP selection) during the reaction phase of the selection process (FIG. 4A, stage I or stage V). This provides a choice of phosphorylating agents that can be used by individual self-phosphorylating deoxyribozymes. Both populations responded positively to the selection constraints, such that ˜35% (NTP) and ˜45% (dNTP) of the DNA populations undergo ligation after six rounds. Further characterization of the active populations and of individual deoxyribozymes provided evidence that catalysis results in the transfer of the (d)NTP γ phosphate to the 5′-terminal oxygen of DNA (see below).

Deoxyribozymes with Diverse Substrate Specificities.

Populations identified after seven rounds (denoted G7) of the NTP and dNTP selections were cloned, and ˜20 representative clones from each pool were sequenced and assayed for catalytic activity. Analysis revealed considerable sequence diversity. However, among five distinct sequence classes identified from the NTP pool, all require GTP for deoxyribozyme function. Likewise, dGTP-utilizing deoxyribozymes comprise three of the four distinct classes isolated from the G7 dNTP pool. The remaining class (dNTP-N1) is a “universal” kinase that can use any of the four dNTPs with similar efficiency.

Though the G7 populations were dominated by GTP- or dGTP-utilizing deoxyribozymes, deoxyribozymes exhibiting specificities for the remaining nucleotide substrates that displayed distinct preferences for each of the four standard NTPs or dNTPs were recovered in a repeat in vitro selection process run in parallel beginning with G2 DNA, in each case making available only the NTPs or dNTPs with base identities A, C, U, or T for use as substrates by each corresponding population.

Two major and four minor classes of ATP-dependent deoxyribozymes were isolated from the NTP G7 population produced by providing ATP exclusively to the selection reaction. Similarly, a single CTP-dependent deoxyribozyme was isolated from the corresponding G8 population produced by supplying only CTP to the reaction mixture. Finally, two classes of deoxyribozymes were isolated from the G10 population generated by the exclusive use of UTP. Although one class required UTP for activity, the other formed a motif with universal substrate specificity that was distinct from the class represented by the universal kinase class.

Since the dNTP G7 population is highly populated with the universal kinase, a more vigorous strategy for the analogous selections of different dNTP specificities was adopted. This included the simulataneous digestion of the population with three restriction enzymes estroying the deoxyribozyme in its double-stranded form using the restriction enzymes (Eag I, BstN I and BsmA I). In addition, the surviving single-stranded population was preincubated with a mixture of the remaining three dNTPs that were not used for positive selection. DNAs that remained unreacted during this “negative” selection were separated from phosphorylated DNAs and subjected to positive selection in the presence of the target dNTP. In this manner, four classes of dATP-dependent deoxyribozymes and two classes of dCTP-dependent deoxyribozymes were isolated from the corresponding G8 pools.

Discrimination Between Ribo- and Deoxyribonucleotides.

A cursory survey of the accumulated sequence data revealed many distinct sequence classes of self-phosphorylating deoxyribozymes. Each class can be further organized into one of several groups based on NTP or dNTP substrate requirements. In addition to the specific groups described above, several individuals that function as “semi-universal” kinases that, for example, use only (d)GTP and (d)CTP or that use (d)GTP and (d)ATP were observed.

Of the nine classes of deoxyribozymes identified in these experiments, only one displayed significant nucleotide discrimination based on the 2′-hydroxyl group. This finding brings into question the structural diversity of DNA and whether the limitations set by this polymer's chemical simplicity preclude it from distinguishing between closely related organic compounds. To examine this issue in greater detail, two parallel selections to isolate deoxyribozymes that use GTP to the exclusion of dGTP, and vice versa, were conducted. After six rounds of selection beginning with the G5 populations of the NTP and dNTP lineages, both resulting populations displayed significant discrimination between GTP and dGTP. The function of two individuals were examined in greater detail. One was >100 fold more active with GTP compared to dGTP, while the other was ˜25-fold more active with dGTP versus GTP. For both deoxyribozymes, the differences in K_{M} values are negligible; however, the maximum rate constants obtained under saturating conditions are unequal. Surprisingly, specificity in each case was not due to differential binding affinity, but was a result of the differences in reactivity of the nucleotides when bound to the deoxyribozyme. This conclusion is supported by the observation that dGTP serves as a competitive inhibitor of the former deoxyribozyme. These and earlier findings (Breaker, 1997 Curr. Opin. Chem. Biol., cited above, and Roth, A. & Breaker, R. R., 1998, Proc. Natl. Acad. Sci. USA. 95, 6027-6031) support the view that DNA has sufficient structural complexity to recognize subtle chemical differences among substrate, cofactor and effector molecules.

A Distinct Class of ATP-Dependent Deoxyribozymes.

All prototypic deoxyribozymes examined operate with k_{cat} values of 10⁻⁴ min⁻¹ or lower and K_{M} values near 1 mM. To further optimize kinetic characteristics, a mutagenized pool based on an ATP-dependent deoxyribozyme was chemically synthesized (Breaker, R. R. & Joyce, G. F., 1994, Trends Biotechnol. 12, 268-275). After five rounds of selection using progressively lower concentrations of ATP and shorter reaction times, a population of variant deoxyribozymes was recovered. Improvements in both catalytic rate and ATP binding affinity were evident upon examination of one variant, a representative clone from the G5 population. The ATP-dependent ribozyme exhibited a k_{cat} of 1.7×10⁻⁴ min⁻¹ and a K_{M} of ˜1 mM, while the variant had a k_{cat} of 5.5×10⁻³ min⁻¹ and a K_{M} of 3.3 μM in the same assay, corresponding to an improvement in catalytic efficiency (k_{cat}/K_{M}) of ˜10,000 fold. Estimating the rate enhancement of ATP hydrolysis in the presence of Ca²⁺ in the absence of enzyme, which has a reported second-order rate constant of ˜25×10⁻⁷ M⁻¹ min⁻¹ at 23° C. (Ramirez, F., et al., 1980, J. Org. Chem. 45, 4748-4752), the rate enhancement (k_{cat}/K_{M})/k_{hydrolysis} for the variant is ˜7×10⁸, indicating that this deoxyribozyme transfers the γ-phosphate from ATP to its 5′-hydroxyl group nearly one billion times faster than the spontaneous hydrolysis of ATP.

The variant was highly selective for ATP versus CTP, GTP and UTP. The apparent K_{D} for ATP, interpreted as the concentration required to for half-maximal deoxyribozyme activity, corresponds with the K_{M} value determined for the same deoxyribozyme. In contrast, the binding constants for the non-cognate NTPs appear to be more than four orders of magnitude lower. As expected, the k_{cat}/K_{M} value for ATP was more than 10,000-fold larger than the k_{cat}/K_{M} values determined for the remaining NTPs. These findings are indicative of a polymer that is of sufficient structural sophistication to exploit differences in molecular composition to achieve precise substrate recognition.

The ATP-dependent deoxyribozyme required both Ca²⁺ and K⁺ for activity. However, the variant functioned at a maximum rate (k_{obs}=0.012 min⁻¹) in the presence of 20 mM Ca²⁺ (apparent K_{D} for Ca²⁺≈5 mM), and remained active in the absence of K⁺. Among the different sequence classes of deoxyribozymes examined, only Ca²⁺ and Mn²⁺ served as divalent metal ion cofactors. Ca²⁺ dependence also has been demonstrated for a recently described capping ribozyme (Huang, F. & Yarus, M., 1997, Proc. Natl. Acad. Sci. USA. 94, 8965-8969), indicating that this divalent ion may be well suited for catalytic roles in certain phosphate transfer reactions. In contrast, the self-phosphorylating ribozymes previously isolated (Lorsch, J. R. & Szostak, J. W., 1994, Nature 371, 31-36) require Mg²⁺ as a cofactor. However, these ribozymes were selected to perform in a reaction mixture that contained Mg₂₊ alone. Although Ca²⁺ is only five fold more effective than Mg²⁺ at promoting ATP hydrolysis (Ramirez, et al., cited above), RNA may also favor Ca²⁺ as a cofactor for self-phosphorylation if presented with the option.

Verification of Self-Phosphorylation by DNA.

The selection protocol was designed to isolate DNAs that acquire a 5′-phosphate group when incubated with nucleoside 5′-triphosphates and that serve as donor oligonucleotides during the subsequent ligation reaction. To verify self-phosphorylation, aAn ATP-dependent class and a dGTP-dependent deoxyribozyme class showed patterns of DNA ligation that were consistent with the acquisition of a 5′-phosphate group at their 5′ terminus in an ATP- or dGTP-dependent manner, respectively. Likewise, successful DNA ligation required the addition of DNA ligase, template and acceptor oligonucleotides, and ATP; requirements that are in agreement with the selection scheme proposed in FIG. 4A.

To further investigate the nature of the deoxyribozyme-mediated reaction, an unlabeled ATP-dependent deoxyribozyme variant was incubated with (γ-³²P)ATP under permissive reaction conditions. The deoxyribozyme became radiolabeled in a time-dependent fashion, and formed a product that comigrated on a polyacrylamide gel with an authentic 5′-phosphorylated DNA prepared with T4 PNK. These findings are consistent with the transfer of the γ-phosphate of ATP to the 5′-hydroxyl group of DNA. Moreover, the rate constant derived for the increase in ³²P-labeled DNA (k_{obs}=4.0×10⁻³ min⁻¹) matched the rate constant obtained by indirectly measuring phosphorylation via DNA ligation.

Comparisons of Kinases made of Protein, RNA and DNA.

Previously, Lorsch and Szostak (1994 Nature, cited above) described the in vitro selection of several self-phosphorylating ribozymes that use ATP-(γS), a thiophosphate-containing analog of ATP. Although the pool designs and selection strategies used in this section of the example were fundamentally different between these two studies, both efforts produced a number of distinct motifs that catalyze the phosphorylation of nucleic acids. In the study, the remaining natural NTP and dNTP substrates were targeted with almost equal effectiveness. It is clear from these results that DNA, like RNA, is capable of forming a variety of sophisticated tertiary structures that effectively promote chemical catalysis.

Perhaps the most important issue is that of catalytic rates and efficiency. The k_{cat} for the optimized variant deoxyribozyme described above is approximately 30-fold less than the k_{cat} for the most active ATP-(γS)-dependent ribozyme isolated (Lorsch & Szostak, cited above, and Lorsch, J. R. & Szostak, J. W., 1995, Biochemistry 34, 15315-15327). The uncatalyzed rate of ATP-γS hydrolysis is up to 10-fold faster than ATP (Herschlag, D., et al., 1991, Biochemistry 30, 4844-4854 and Breslow, R. & Katz, I., 1968, J. Am. Chem. Soc. 90, 7367-7377). Therefore the differences in chemical rate enhancements between the two polymers are negligible. Interestingly, the K_{M} for ATP exhibited by the variant is more than 10-fold improved from that of the best kinase ribozymes. As a result, the catalytic efficiencies of the DNA and RNA enzymes are nearly equivalent. The k_{cat}/K_{M}⁻¹ values for the variant and “Kin.25” (Lorsch & Szostak, 1994 Nature, cited above) are 3,600 M⁻¹min⁻¹ and 7,900 M⁻¹min⁻¹, respectively. In addition, the K_{M} of the deoxyribozyme is more than 10-fold improved compared to the K_{M} for ATP exhibited by T4 PNK (Lillehaug, J. R. & Kleppe, K., 1975, Biochemistry 14, 1221-1225). However, T4 PNK has a k_{cat} of ˜25,000 min⁻¹, which equates to a catalytic efficiency (˜6×10⁸ M⁻¹min⁻¹) that is approximately five orders of magnitude greater than the variant.

### Preparation of DNA Adenylases

Twelve classes of deoxyribozymes that promote an ATP-dependent “self-capping” reaction (illustrated in FIG. 1B) were isolated by in vitro selection from a random-sequence pool as described above and in detail by Li, et al. (2000 Biochemistry, cited above). Each deoxyribozyme catalyzed the transfer of the AMP moiety of ATP to its 5′-terminal phosphate group, thereby forming a 5′,5′-pyrophosphate linkage. An identical DNA adelylate structure was generated by the T4 DNA ligase during enzymatic DNA ligation. A 41-nucleotide class 1 deoxyribozyme required Cu²⁺ as a cofactor and adopted a structure that recognized both the adenine and triphosphate moieties of ATP or dATP. The catalytic efficiency for this DNA, measured at 10⁴ M⁻¹min⁻¹ using either ATP or dATP as substrate, was similar to other catalytic nucleic acids.

In Vitro Selection of Self-capping Deoxyribozymes.

The phosphoanhydride exchange reaction depicted in FIG. 1B is expected to be the most likely route by which the desired capped structure can be formed. Specifically, a deoxyribozyme uses an oxygen on its own 5′-phosphate group to attack the phosphorus center of the α-phosphate of ATP. Considering that displacement of the pyrophosphate leaving group is very favorable, the reaction could proceed either via an S_{N}1 or an S_{N}2-like mechanism.

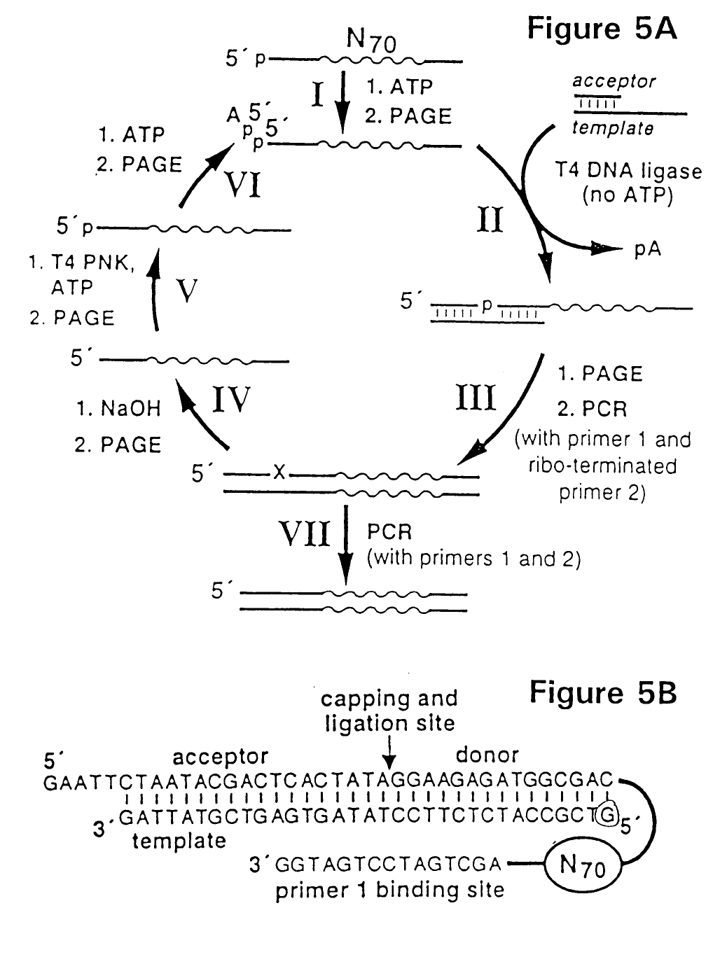

To isolate DNAs that form the desired 5′,5′-pyrophosphate cap in the presence of ATP, the selection scheme shown in FIG. 5A was employed. Analogous to the methods used in the previous section, this strategy exploits the fact that T4 DNA ligase forms a 5′,5′-AppDNA structure that is an essential intermediate of the enzymatic ligation of DNA. If T4 DNA ligase is not supplied with ATP, then only DNAs that carry a pre-formed 5′,5′-pyrophosphate cap are available for ligation. In the selection scheme, individual DNAs from the random-sequence pool were ligated to an acceptor oligonucleotide by T4 DNA ligase only if the molecules acquire the appropriate 5′,5′-pyrophosphate cap when pre-reacted in the presence of ATP. Therefore, self-capping DNA was selectively ligated to an acceptor molecule by T4 DNA ligase, which facilitated their separation by PAGE and their selective amplification by PCR.

The initial DNA population (denoted G0) was comprised of approximately 10¹⁴ individual 100mers, each carrying a 70-nucleotide random-sequence domain flanked on each side by specific primer-binding sites (FIG. 5B). Each DNA molecule in the population also carried a 5′-phosphate group. Initially, the 5′-phosphorylated population of DNAs was incubated with 1 mM ATP for 20 hr and the resulting reaction products were subjected to the selective-amplification process as detailed in FIG. 5A. No significant capping activity was observed for the first iterations of selective amplification as determined by the fraction of DNA pool that was ligated by T4 DNA ligase. However, more than 5% of the population generated after six rounds of selection was joined to the acceptor oligonucleotide. By the ninth round, nearly 15% of the DNA population was joined by T4 DNA ligase despite the absence of ATP in the ligase reaction.

To favor the isolation of DNAs with improved characteristics, more stringent selection conditions were created by reducing the concentration of ATP to 100 μM and by reducing the incubation time to 1 hr. In addition, the DNA populations at rounds 10 and 15 were subjected to a hypermutagenic version of PCR (Vartanian, J.-P., et al., 1996, Nucleic Acids Res. 24, 2627-2631) to provide additional sequence diversity. The DNA at the twenty-first round exhibited significantly improved ligation characteristics, with nearly 20% of the population becoming ligated by T4 DNA ligase in the absence of ATP. Additional rounds of selection using more demanding selection conditions did not provide further improvement in the characteristics of the population.

The DNA population at G22 was cloned and 40 individuals were sequenced. At least 12 distinct classes of DNAs were identified based on their unique sequence composition. In each case, the impact that mutagenesis has had on the population is evident by the variable length of the original random-sequence domain and by the mutations that accumulated in the 5′ donor domain that was held constant in the design of the original construct. Five classes were arbitrarily chosen for preliminary examination, and all exhibited ATP-dependent self-capping activity as determined by the ligation-PAGE method employed for in vitro selection.

Class 1 Self-Capping Deoxyribozyme.

The dominant sequence among the cloned DNAs was a class denominated as 1 and, therefore, this class of DNAs was chosen for study in greater detail. A reselection based on class 1 DNAs was conducted to further optimize its activity and to generate an artificial phylogeny of deoxyribozyme sequence variants. Mutations were introduced throughout the original random-sequence domain again using hypermutagenic PCR. Numerous sequence variants that display activity were isolated after four rounds of selection. Upon examination of the variants, a high frequency of observed random mutations were tolerated by the DNA in the region spanning nucleotides 28-68 of the original random-sequence domain. Almost without exception, however, mutations were not tolerated in the first 27 nucleotides. The most frequent variation in this highly-conserved domain is a G to A mutation at position 12. These results suggest that the highly conserved nucleotides 1-27 are critical for deoxyribozyme function while the remaining variable region might be superfluous for catalytic function.

Interestingly, mutations also had occurred in the donor domain for class 1 deoxyribozymes. This is somewhat unexpected for two reasons. First, this domain was not mutated in the initial random-sequence pool, nor was it intentionally subjected to mutation during the reselection. Second, the base pairing potential of the donor domain cannot be substantially altered during selection because it must be recognized by the template oligomer that is used during ligation by T4 DNA ligase. However, variant deoxyribozymes were found to carry up to four mutations compared to the original donor sequence, indicating that at least a portion of this domain is not essential for efficient deoxyribozyme function.

A Minimized Class 1 Deoxyribozvme.

An artificial phylogeny was used as a guide to design truncated deoxyribozymes in an effort to define the minimum contiguous class 1 DNA that retains full catalytic activity. Synthetic DNAs corresponding to truncations at nucleotides 24-28 were 5′ 32P-labeled and individually incubated under self-capping reaction conditions. PAGE separation of the self-capping reaction products prior to treatment with T4 DNA ligase revealed that certain DNAs yielded a product that was indicative of the addition of a 5′,5′ pyrophosphate cap. These results demonstrate that a DNA terminating at G27 encompasses the minimal essential structure for full activity, while shorter DNAs that terminate at positions 24, 25 or 26 do not exhibit catalytic activity. Therefore, a total of 41 nucleotides from the 5′ terminus of the original class 1 sequence encompass the minimum contiguous deoxyribozyme domain.

Product Verification using Class 1 Deoxyribozymes.

The observation that T4 DNA ligase accepted deoxyribozymes as donors in a ligation reaction only upon pre-incubation with ATP is consistent with the view that deoxyribozymes generate the intended 5′,5′-pyrophosphate cap. It was also observed that rapid dephosphorylation of the pre-reacted deoxyribozymes by alkaline phosphatase occured only if the capped structure had been subjected to periodate oxidation followed by β-elimination (Nadeau, J. G., et al., 1984, Biochemistry 23, 6153-6159). This treatment was expected to selectively remove the adenosine moiety from the pyrophosphate structure only if the 2′- and 3′-hydroxyl groups of the nucleoside are displayed at the terminus of the deoxyribozyme, thereby making the 5′ ³²P moiety available for removal by phosphatase. The observation that periodate oxidation/β-elimination treatment is required for efficient phosphatase activity also is consistent with the deoxyribozyme-mediated formation of the predicted cap structure.

To provide additional support for the DNA-mediated formation of a 5′,5′-pyrophosphate cap, an “α-³²P acquisition” analysis wherein different radiolabeled forms of ATP were used to assess the structure of the modification. Unlabeled 41mer class 1 deoxyribozymes corresponding to a parent sequence were independently incubated in the absence of ATP, or in the presence of (γ-³²P)-, (α-³²P)-, or unlabeled ATP (10 μM final concentration). Analysis of the DNA products by PAGE yielded the expected capped DNA when any form of ATP was supplied (as visualized using ethidium bromide staining followed by UV translumination). As expected, the absence of ATP precluded deoxyribozyme activity. However, only the product derived from the deoxyribozyme reaction conducted in the presence of (α-³²P)ATP was visible by autoradiography of the same gel. This indicated that the DNA molecule incorporates the α-phosphate from ATP as a consequence of its catalytic activity. The acquisition of α-phosphate rather than γ-phosphate is consistent with the expected capping mechanism depicted in FIG. 5A.

The results described above, however, do not rule out the possibility that the deoxyribozyme acquires both the α and β phosphates of ATP by attacking the phosphate at the β position, with concomitant expulsion of the γ phosphate. To examine this possibility, the ATP analogs β,γ-methylene ATP and α,β-methylene ATP were tested. Each carries a cleavage resistant methylene linkage in place of the normal phosphoanhydride linkage. It was found that while β,γ-methylene ATP can be used by the deoxyribozyme, α,β-methylene ATP cannot serve as a substrate. This result is consistent with a mechanism whereby the phosphorus center of the α-phosphate group of ATP is attacked by an oxygen atom of the 5′ phosphate of the deoxyribozyme. This would result in release of pyrophosphate and formation of the expected 5′,5′-pyrophosphate cap.

Reaction Parameters for Optimal Deoxyribozyme Function.

To establish the optimal reaction conditions for class 1 DNAs, the influences of pH, ionic strength and cofactor dependence of the 41mer deoxyribozyme were examined. Although the selection reactions were conducted at pH 7.0, the 41mer displayed near full catalytic activity over a broad pH range with a maximum of 3×10⁻³min⁻¹ at pH 6.5. The deoxyribozyme required both Cu²⁺ and Mg²⁺ as cofactors, although it is not yet clear precisely what roles these divalent metals play in the self-capping reaction. Mg²⁺ can be replaced with Ca²⁺ with near equal efficiency. Cu²⁺, which binds to the deoxyribozyme with an apparent K_{d} of ˜3 μM, is known to serve as an essential cofactor for several other deoxyribozymes (Carmi, et al., 1996 Chem. Biol, Carmi, et al., 1998 P.N.A.S., and Cuenoud & Szostak, 1995 Nature, cited above). However, it is important to note that Cu²⁺ becomes strongly inhibitory at concentrations above 20 μM, in accordance with its ability to interact non-specifically with DNA and disrupt structure at concentrations in excess of 10 μM (Eichhorn, G. L., & Shin, Y. A., 1968,. J. Am. Chem. Soc. 90, 7323-7328 and Rifkind, J. M., et al., 1976, Biopolymers 15, 1879-1902). As a result, the concentration of deoxyribozyme and its Cu²⁺ cofactor must be tightly controlled in order to avoid deoxyribozyme inactivation.

Perhaps the most peculiar behavior of this deoxyribozyme was its requirement of Na⁺ for full catalytic activity. A minimum of 0.25 M NaCl was required for the deoxyribozyme to reach maximum catalytic activity, which might be expected if its role is exclusively to establish the ionic strength of the reaction mixture. However, K⁺ failed to fully substitute for Na⁺ in this capacity and even became somewhat inhibitory at concentrations above 0.1 M. Likewise, neither Li⁺ nor NH₄⁺ could substitute for Na⁺ in the reaction. The inhibitory effect of K⁺ indicated that K+ might selectively bind to the DNA and favor the formation of structures that compete with the active structure of the deoxyribozyme.

Substrate Recognition of ATP and its Analogs.

Class 1 deoxyribozymes exhibited classical Michaelis-Menten kinetics towards its ATP substrate, which had a K_{M} of ˜500 nM. Under saturating concentrations of ATP, the 41mer deoxyribozyme displayed a k_{cat} of 4.8×10⁻³ min⁻¹ and a k_{cat}/K_{M} of ˜10⁴ M⁻¹min⁻¹ under optimal reaction conditions. Interestingly, the deoxyribozyme was equally efficient when dATP is substituted for ATP. This indicates that the 2′-hydroxyl group of ATP does not contribute significantly to the binding and catalysis by class 1 DNAs.

To further establish the chemical groups of the substrate that are critical for molecular recognition and chemistry, the substrate characteristics of a series of nucleoside triphosphates and ATP analogues were examined. As expected, the catalytic activity of the 41mer can be observed upon incubation with either ATP or dATP. In contrast, the presence of an intact ribose ring and the 3′-hydroxyl group were critical for catalytic activity. In addition, the adenine base moiety of ATP carried key determinants of substrate specificity. This was evident by the fact that neither the three remaining NTPs nor the analogues ITP or XTP are accepted as substrate by the deoxyribozyme.

The activity of substrate analogs with changes to the triphosphate moiety of ATP revealed important characteristics of the intended phosphoester transfer reaction. First, the deoxyribozyme required a triphosphate structure for catalysis, as adenosine-5′ diphosphate failed to serve as a substrate. Second, the use of sulfur to replace single oxygen atoms on the γ- and α-phosphate groups resulted in diminished catalytic activity. This suggested that these positions might form contacts with the deoxyribozyme that are critical for catalytic function. Sulfur substitution within the α-phosphate group also might reduce the rate of DNA capping by perturbing the electronic characteristics of the target phosphorus center. This might explain why the α-thiophosphate substitution had a greater impact on the catalytic rate than did the γ-thiophosphate substitution. Third, independent replacements of the two oxygen bridges that link the phosphates with methylene bridges had different outcomes. The deoxyribozyme exhibited reduced but measurable capping activity when supplied with the (β-γ-methylene) ATP analog. However, no activity was detected when the deoxyribozyme was incubated with the (α-β-methylene) ATP analogue. The methylene bridges are expected to resist enzymatic cleavage by enzymes, made either of protein or nucleic acid, that catalyze the cleavage of phosphoanhydride bonds. Therefore, the observation that the ATP analog with the α-β-methylene bridge was not a substrate indicates that the phosphorus center at the α position is the target for the reaction and that the phosphoanhydride linkage between the α and β phosphates is cleaved.

Catalytic Performance of the Capping Deoxyribozyme.

The minimized class 1 deoxyribozyme brings about a modest catalytic rate constant (k_{cat}) of ˜5×10⁻³ min⁻¹, but exhibits a high affinity for the external ATP substrate (K_{M}=0.5 z,900 M). As a result, the catalytic efficiency (k_{cat}/K_{M}) of this deoxyribozyme approaches 10⁴ M⁻¹min⁻¹. This catalytic efficiency is comparable to many of the natural ribozymes and other selected ribozymes and deoxyribozymes that use external substrates of similar size and nature. For example, k_{cat}/K_{M} values ranging from 10³ M⁻¹min⁻¹ to 10⁴ M⁻¹min⁻¹ for nucleic acid enzymes such as the Tetrahymena self-splicing intron (Bass, B., & Cech, T. R., 1984, Nature 308, 820-826), a self-phosphorylating ribozyme (Lorsch & Szostak, 1994 Nature, cited above), a self-capping ribozyme (Huang & Yarus, 1997 P.N.A.S., cited above), and the self-phosphorylating deoxyribozyme described in the previous section. Unfortunately, it is difficult to experimentally determine the rate constant for the uncatalyzed capping of DNA by ATP due to its slow rate. However, the overall rate enhancement achieved by the class 1 deoxyribozyme can be estimated by comparing its rate constant to that of the uncatalyzed rate of ATP hydrolysis. Hydrolysis of ATP has a reported second-order rate constant of ˜5×10⁻⁷ M⁻¹min⁻¹ in the presence of Mg²⁺ (Ramirez, et al., 1980 J. Org. Chem., cited above). Therefore, the rate enhancement (k_{cat}/K_{M})/k_{hydroysis} for the class 1 self-capping deoxyribozyme is ˜2×10¹⁰, indicating that this deoxyribozyme transfers AMP from ATP to its 5′-phosphate group more than 10 billion times faster than the corresponding spontaneous hydrolysis of ATP.

### Preparation of Conjoined Polynucleotides

Kinase and adenylase deoxyribozymes isolated as described above can be used together in a reaction mixture to self-phosphorylate and self-adenylate in the presence of ATP. Alternatively, the DNA functional domains are coupled using DNA ligase in combination with a template oligonucleotide to form the construct illustrated in FIG. 2.

### Example 2

This example provides a ligase deoxyribozyme that catalyzes the reaction illustrated in FIG. 1C, and constructs useful in DNA cloning which are illustrated in FIG. 3 by linking a DNA construct comprising a phosphorylase and a adenylase domain isolated in Example 1 with a ligase domain as illustrated in FIG. 3.

### Preparation of DNA Ligases

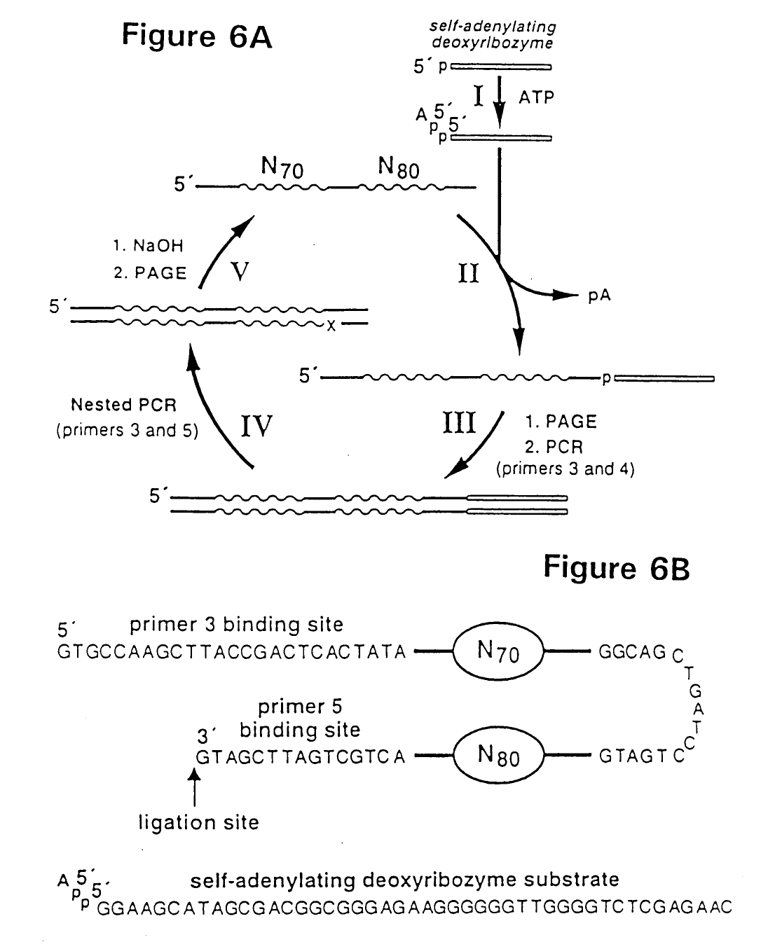

The isolation of self-ligating deoxyribozymes that mimic the last step of T4 DNA ligase can be achieved using the selection strategy depicted in FIG. 6A. Again, the principles of the selective-amplification process are similar to those discussed above. However, the iterative selection protocol is somewhat more simplified. Specifically, DNAs that couple with a chemically activated substrate DNA are separated from unreacted DNAs using PAGE. Therefore, the selection strategy relies on only three basic principles. First, DNAs must be present within the original random-sequence population that possess the ability to form a new 3′-5′-phosphodiester linkage with the target DNA at the expense of a 5′,5 -pyrophospate linkage with an adenyl moiety (FIG. 1C). Second, PAGE is used to separate ligated from unligated DNAs, thereby facilitating the selective recovery of self-ligating deoxyribozymes. Third, PCR is used to selectively amplify the DNAs that survive the preceding steps of the selection process.

Strategy for the In Vitro Selection of Self-lipating Deoxyribozymes that Use ATP.

In preliminary experiments using T4 DNA ligase, the selection strategy outlined in FIG. 6A was used to isolate a new class of deoxyribozymes that mimic the last step of T4 DNA ligase. A 208-nucleotide DNA construct, depicted in FIG. 6B, was used to isolate self-ligating deoxyribozymes. This construct differed in two respects from the construct used in preliminary experiments. First, the random-sequence content was increased to 150 nucleotides, although lesser random content would probably be sufficient to provide new deoxyribozymes. Second, template and acceptor oligonucleotides used in preliminary experiments were rendered unnecessary because ligation by T4 DNA ligase is no longer needed for the selection.

Another significant distinction of this selection was that a pre-charged DNA is used as substrate. In this protocol, a self-adenylating deoxyribozyme was used as the substrate for in vitro selection of ligase deoxyribozymes. The self-adenylating deoxyribozyme is reacted with ATP, purified, and stored at −20° until needed for the selection reaction. Those DNAs that form a new 3′-5′-phosphodiester linkage with the self-adenylating deoxyribozyme substrate (FIG. 1C) are extended by 46 nucleotides, which reflects the size of the DNA substrate. The newly formed ˜254-nucleotide product is isolated by PAGE and the recovered DNAs are selectively amplified by PCR. In this case, regeneration of the DNA population uses both nested PCR and embedded RNA strategies.

Synthesis of oligonucleotides.

The DNA construct cannot be efficiently synthesized by standard automated chemical synthesis. Therefore, larger constructs such as this can be assembles from smaller synthetic DNAs by using T4 DNA ligase and the appropriate DNA template. The DNA construct depicted in FIG. 6B was assembled from two fragments by employing a template DNA that is complementary to the defined nucleotide sequence intervening the two random-sequence domains. T4 DNA ligase was used to catalyze the joining reaction using the manufacturer's recommended conditions.

Pre-adenylated deoxyribozymes that serve as the substrate for the selection were prepared under reaction conditions similar to those used for their in vitro selection. For example, large scale preparation of adenylated DNA substrate is initiated by phosphorylation of 30 nmoles of self-adenylating deoxyribozyme (FIG. 6B) using T4 polynucleotide kinase according to the manufacturer's directions. The phosphorylated DNA is recovered by precipitation with ethanol, and pelleted DNA is resuspended in 1 mL dH₂O, heated at 90° C. for 3 min, then allowed to cool to 23° C. The self-adenylation reaction was initiated by the addition of 14 mL 2× self-adenylation buffer (100 mM HEPES (pH 7.0 at 23° C.), 500 mM NaCl, 20 mM MgCl₂), 28 μl 10 mM CuCl₂, 56 μl 50 mM ATP and 12.9 mL dH₂O. The reaction was incubated at 23° C. for three days, then terminated by the addition of 560 μl 0.5 M EDTA, 2 mL 3 M NaOAC, and 80 mL 100% ethanol. The resulting precipitate was recovered by centrifugation and the pelleted DNA is resuspended in 50 μl dH₂O and 50 μl 2× PAGE loading buffer. The adenylated DNA product was separated from unreacted DNA by denaturing 15% PAGE and recovered from the gel by crush/soak elution.

The Deoxyribozyme Selection Reaction.

An in vitro selection reaction containing 200 pmol of the DNA construct depicted in FIG. 6B provided approximately 10¹⁴ different DNA sequences that can be screened for DNA ligase deoxyribozymes. The selection reaction was prepared by combining 2 μl DNA construct with 20 μL pre-adenylated deoxyribozyme substrate and 158 μl of dH₂O. The mixture was heated to 90° C. for 1 min, cooled to room temperature, and combined with 20 μL of 5× selection buffer (250 mM HEPES (pH 7.0 at 23° C.), 1000 mM KCl, 50 mM MgCl₂, 25 mM CaCl₂, 5 mM MnCl₂) to initiate the selection reaction. The mixture was incubated at 23° C. for 48 hr, and the reaction was processed as described above. Since the DNAs being sought are self-ligating, treatment of the population with T4 DNA ligase is not necessary. Purification of the resulting 254-nucleotide DNAs was carried out by PAGE as described above. DNA recovered by crush/soak elution of the excised gel was precipitated with ethanol from a sample that was supplemented with 5 μL each of 20 pmole μL⁻¹ primer 3 (SEQ ID NO: 10) and 20 pmol μL⁻¹ primer 4 (SEQ ID NO: 11). Subsequent selection reactions can be reduced to 1/10^{th} scale.

PCR Amplification and Regeneration of the DNA Population.

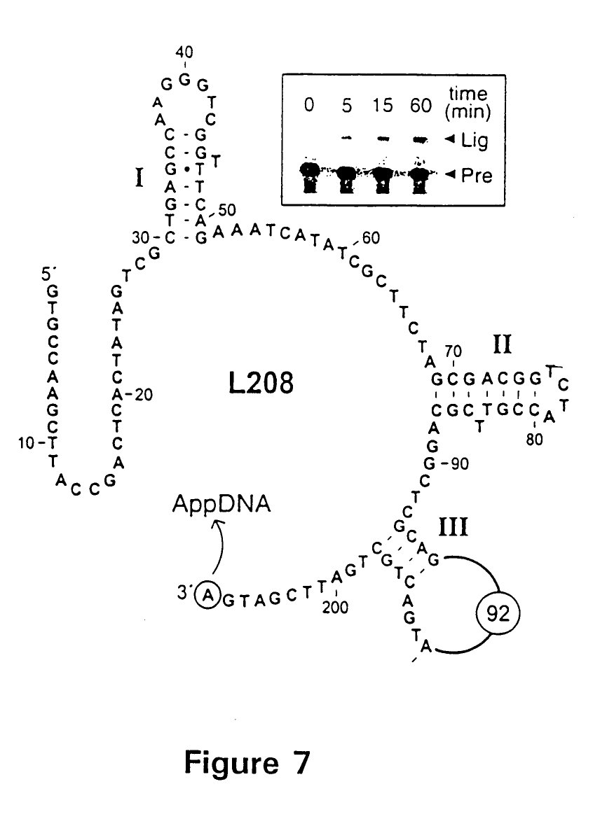

Initial amplification of the selected DNAs was conducted as described above. However, regeneration of the DNA population for the next round of selection requires the application of nested PCR, wherein the ligated DNA domain (the self-adenylated DNA substrate) is eliminated from the construct. To achieve this, a second PCR reaction was prepared with 1 μL of the preceding PCR reaction, 72 μL dH₂O, 2.5 μL 20 pmoles μL⁻¹ primer 3, 2.5 μL 20 pmoles μL⁻¹ primer 5 (SEQ ID NO: 12), 10 μL10× PCR Buffer, 10 μL 10× PCR dNTPs, 1 μL α-³²P [GTP], 1 μL 5 U μL⁻¹ Taq DNA polymerase, and subjected to 10 thermocycles as described above. The use of primer 5 serves two purposes. First, this primer is complementary to the original terminus of the DNA construct and therefore yields DNA amplification products that are truncated at the original 3′ terminus. Second, primer 5 is terminated with a ribonucleotide at its 3′ end, which facilitates cleavage of the (−) strand upon treatment with alkali. In this manner, (+) strand DNAs of original length can be purified by PAGE. This selection and amplification process is reiterated until satisfactory activity is exhibited by the population. The sequence and secondary structure of a self-ligating DNA that requires the action of a self-phosphorylating DNA for its function is set out in FIG. 7.

### Preparation of Conjoined Polynucleotides

The ligase of this example is fused to the kinase-adenylase construct prepared in Example 1 using the method set out in Example 1 to prepare the DNA construct illustrated in FIG. 3, which exhibits kinase/adenylase/ligase activity. A self-circularizing DNA construct is prepared using existing catalytic components by arranging them such that the capped 5′ end and the 3′ ligation site are on the same molecule. Upon activation by ATP, this serves as a template for rolling-circle replication.

### Example 3

Some conjoined polynucleotides of the invention are immobilized on a solid solid support, using chemical bonding or adsorption, for use as chromatographic matrices, biochips, and molecular assemblies in this example. One is prepared by immobilizing the kinase and adenylase deoxyribozymes of Example 1 on a solid support; similar activity is obtained by immobilizing the kinase/adenylase construct illustrated in FIG. 2 on a solid support. Another is prepared by immobilizing the kinase, adenylase, and ligase deoxyribozymes prepared in Example 2 on a solid support, immobilizing the kinase/adenylase construct illustrated in FIG. 2 and the ligase deoxyribozyme on a solid support, or the kinase/adenylase/ligase construct illustrated in FIG. 3 on a solid support. DNAs that catalyze the reactions necessary to couple to specific compounds, e.g., by use of the conjoined kinase/adenylase complex or construct with a nucleophile, are fused to the catalytic domains or constructs, so that the deoxyribozymes or constructs immobilize themselves.

### Example 4

This example illustrates the use of an activated construct of Example 1 containing a phosphorylase domain and an adenylase domain (FIG. 2) to ligate to proteins and other biological targets that are not nucleotides or oligonucleotides.

The kinase/adenylase construct is employed to couple the undecapeptide called “substance P”, an important neurotransmitter and neuromodulator in the central and peripheral nervous system of mammals, to a support. This signaling peptide is made in large quantities and modified with tyrosine and cysteine to introduce a reactive nucleophile and allow selective separation by thiol-mediated immobilization, respectively. Immobilized DNA-peptide conjugates are recovered from a thiophilic matrix by elution with DTT. Recovered DNAs are amplified by PCR as described previously (Li & Breaker, 1999 Curr. Opin. Struct. Biol., Li & Breaker, 1999 P.N.A.S., and Li, et al., 2000 Biochemistry, cited above).

Similarly, the kinase/adenylase construct is employed to couple the flurorochrome carboxyfluoroscein diacetate succinimyl ester to the DNA construct using the methodology set out in Example 1. Recovered DNAs are amplified using standard means to provide self-labeling DNA.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the claimed components and steps in any sequence which is effective to meet the objectives there intended, unless the context specifically indicates the contrary.

1-20. (canceled)

21. A method of isolating a class of self-ligating deoxyribozymes comprising the steps of:
a) identifying DNAs within a random-sequence population of DNAs that possess an ability to form a new 3′-5′-phosphodiester linkage with the DNA at the expense of a 5′,5′-pyrophosphate linkage with an adenyl moiety;
b) separating DNAs that couple with a chemically activated substrate DNA from unreacted DNAs to facilitate selective recovery of self-ligating deoxyribozymes using PAGE; and
c) selectively amplifying the DNAs that survive step b) using polymerase chain reaction (PCR).

22. The method according to claim 21, wherein the nucleotide DNA construct comprises a self-adenylated dexoxyribozyme comprising SEQ ID NO:4, SEQ ID NO:5, SEQ ID NO: 6, and SEQ ID NO:7.

23. A method of isolating a class of self-phosphorylating deoxyribozymes by in vitro selection comprising the steps of:
(a) incubating a population of synthetic DNAs that carry a random sequence domain with a source of phosphate;
(b) combining the DNAs of step (a) with excess acceptor and template oligonucleotides;
(c) treating the DNAs with T4 DNA ligase in the presence of ATP to produce ligated DNAs;
(d) isolating the ligated DNAs produced in step (c) using PAGE;
(e) selectively amplifying the ligated DNAs by polymerase chain reaction (PCR) to produce a double stranded PCR product;
(f) treating the double stranded PCR product with sodium hydroxide to cleave the isolated RNA linkage and produce single stranded DNAs;
(g) purifying the single-strand DNAs by PAGE;
(h) using the purified single-stranded DNAs to initiate a subsequent round of selection;
(i) repeating steps (a) through (h) until the DNA populations exhibits a desired level of catalytic activity;
(j) amplifying DNA populations from the final round of selection by PCR to facilitate cloning of the selected DNA populations.

24. A method of isolating a class of deoxyribozymes that form a 5′5′-pyrophosphate cap in the presence of ATP, the method comprising the steps of:
(a) incubating a pool of synthetic DNAs, wherein each DNA carries a random sequence domain and a 5′-phosphate, in a selection buffer containing ATP;
(b) separating the DNAs from ATP by PAGE;
(c) combining the DNAs with excess acceptor and template oligonucleotides and treating the DNAs with T4 DNA ligase in the absence of ATP to produce ligated DNAs complexed with acceptor and template oligonucleotides;
(d) isolating the ligated DNAs by PAGE and selectively amplifying the ligated DNAs by polymerase chain reaction (PCR) to produce a double-stranded PCR product;
(e) treating the double-stranded PCR product with sodium hydroxide to cleave a single RNA linkage and separating the resulting single-stranded DNAs by PAGE;
(f) phosphorylating the single-stranded DNAs with T4 PNK in the presence of ATP and purifying the DNAs by PAGE;
(g) using the phosphorylated DNAs to initiate the next round of selection;
(h) repeating steps (a) through (g) until a desired level of catalytic activity is achieved; and
(i) amplifying ligated DNAs from the final round of selection with PCR to facilitate cloning of the selected DNA populations.

25. A method of isolating self-phosphorylating DNAs from a population of (+) strand random sequence DNAs comprising the steps of:
(a) incubating the population of (+) strand random sequence DNAs with a chosen source of phosphate to produce a phosphorylated DNA product;
(b) combining the phosphorylated DNA product with an acceptor-template combination that hybridizes to a donor domain of each DNA in the population of (+) strand random sequence DNAs such that a 5′ end of the donor domain is adjacent to a 3′ hydroxyl group of the acceptor oligomer with the templated complex, wherein only those random sequence DNAs that have acquired a 5′-phosphate group serve as a substrate for a T4 DNA ligase;
(c) joining each phosphorylated DNA to the acceptor oligonucleotide using T4 DNA ligase;
(d) isolating ligated DNAs by PAGE and amplifying the ligated DNAs using polymerase chain reaction (PCR);
(e) incubating the resulting (+) strand DNAs to initiate a subsequent round of selection;
(f) repeating steps (a) through (e) until the DNA population exhibits a desired level of catalytic activity.
(g) amplifying the DNA population of step (f) by PCR and subjecting the resulting double-stranded DNAs to cloning and sequencing to examine individual kinas deoxyribozymes.
